(19) United States
(12) Patent Application Publication
(10) Pub. No.: US 2013/0121961 A1
(43) Pub. Date: May 16, 2013

(54) TREATMENT MODALITIES TO PREVENT OR TREAT LOSS OF CARDIOVASCULAR FUNCTION IN AGING HUMANS

(71) Applicant: The Johns Hopkins University, Baltimore, MD (US)

(72) Inventors: Gautam Sikka, Baltimore, MD (US); Dan E. Berkowitz, Baltimore, MD (US); Lili Ayala Barouch, Ellicott City, MD (US); Jeremy D. Walston, Baltimore, MD (US)

(73) Assignee: THE JOHNS HOPKINS UNIVERSITY, Baltimore, MD (US)

(21) Appl. No.: 13/674,197

(22) Filed: Nov. 12, 2012

Related U.S. Application Data
(60) Provisional application No. 61/558,675, filed on Nov. 11, 2011.

(51) Int. Cl. C12Q 1/02

(52) U.S. Cl. CPC C12Q 1/025
USPC 424/85.2; 435/29

(57) ABSTRACT

Provided herein are methods for treating, preventing or reducing age related vascular stiffness and impaired cardiovascular function in a subject comprising administering to the subject a therapeutic amount of IL-10 or an IL-10 agonist or pharmaceutical compositions comprising the same. Also included herein are methods for determining whether a biologically active agent can treat, prevent or reduce age related vascular stiffness and impaired cardiovascular function using an in vitro model in a IL-10 knockout IL-10(tm/tm) mouse which lacks IL-10 function.

### REFERENCE TO RELATED APPLICATIONS

This application claims the benefit of U.S. Provisional Patent Application No. 61/558,675, filed on Nov. 11, 2011, which is hereby incorporated by reference for all purposes as if fully set forth herein.

### STATEMENT OF GOVERNMENTAL INTEREST

This invention was made with U.S. government support under grant no. HL105296-02. The U.S. government has certain rights in the invention.

### BACKGROUND OF THE INVENTION

Aging is inevitable, yet its physiologic consequences are, to some degree, modifiable. Cardiovascular (CV) dysfunction is the final common pathway of many acquired disease states and hence the most common cause of age-related deaths in the United States. Frailty is a geriatric syndrome of late-life vulnerability to adverse outcomes and early mortality associated with declines in multiple physiological systems, the activation of inflammatory pathways, skeletal muscle decline, and subclinical cardiovascular disease. Given that frailty is such an important marker of adverse outcomes, the identification of etiological pathways that influence frailty-related vulnerability will greatly facilitate the development of improved risk assessment and better preventive and treatment modalities.

Interleukin (IL) 10 was originally demonstrated to be an anti inflammatory product of T helper 2 cells. Genetic deletion of IL-10 in mice leads to a series of IL-10 associated pathologies. An increased risk of developing enterocolitis and colorectal cancer, inflammatory bowel disease, development of osteopenia, decreased bone formation, mechanical fragility of long bones, and exacerbation of fatigue and motor deficits have been demonstrated in IL-10 deficient mice. This phenotype is consistent with frail older humans.

Further studies have shown that IL-10 inhibits LDL/Ox-LDL dependent monocyteendothelial interaction thereby inhibiting atherogenesis and hence preventing the development of atherosclerotic plaque in mice. Furthermore, plasma IL-10 levels have been shown to decrease in patients following myocardial infarction. Additionally, data demonstrates that plasma IL-10 levels are directly correlated with good prognosis and remain an independent predictor of long-term adverse cardiovascular outcomes in Acute Coronary Syndromes. IL-10 levels also have a strong inverse correlation with stroke mortality, as shown in the Leiden 85-Plus study.

Therefore, there still exists an unmet need to develop mammalian models of cardiovascular frailty and their use in identifying new treatment modalities to prevent or treat loss of cardiovascular function in aging humans.

### SUMMARY OF THE INVENTION

In accordance with an embodiment, the present invention provides a method to reduce, prevent, or delay age-related vascular stiffness in a subject comprising administrating to the subject a pharmaceutical composition comprising a therapeutically effective amount of IL-10, or an IL-10 receptor agonist.

In accordance with another embodiment, the present invention provides a method for screening biologically active agents which modulate IL-10 related effects on cardiovascular tissue comprising: a) providing test cardiovascular tissue from a IL-10(tm/tm) mouse and control cardiovascular tissue from a WT mouse; b) contacting the biologically active agent with both the test and control cardiovascular tissue for a sufficient period of time; c) measuring the effect of the biologically active agent on both the test and control cardiovascular tissue; wherein the effect being measured is selected from the group consisting of vasorelaxation, mean arterial blood pressure, pulse wave velocity, COX-2 mRNA expression, iNOS mRNA expression, left ventricular end-systolic diameter (LVESD) ejection fraction (EF), intraventricular septal thickness at end of diastole/left ventricular posterior wall thickness at end of diastole (IVSD/LVPWD) ratio, left ventricular (LV) mass, myocyte size and isovolumic relaxation time (IVRT); d) comparing the effect of the biologically active agent on both the test and control cardiovascular tissue, wherein when the effect of the biologically active agent on the test tissue is significantly different from the effect of the biologically active agent on the control tissue, identifying the biologically active agent as modulating IL-10 related effects on cardiovascular tissue.

### BRIEF DESCRIPTION OF THE DRAWINGS

FIG. 1A Acetylcholine (ACH) dependent vasorelaxation recorded via force tension myography, is no different in young Interleukin (IL)-10(tm/tm) and wild type (WT) mouse aortas. FIG. 1B Example tracing: young IL-10(tm/tm) aorta in the presence and absence of indomethacin (above) and young WT aorta in the presence and absence of indomethacin (below). FIG. 1C Endothelial dependent vasorelaxation is markedly diminished in old IL-10(tm/tm) mice compared to old WT controls. Insets within (FIGS. 1A, 1C) represent diminishing ACH dependent relaxation at a 1 μM in IL-10(tm/tm) but not WT aorta. FIG. 1D Example tracing: old IL-10(tm/tm) aorta in the presence and absence of indomethacin (above) and old WT aorta in the presence and absence of indomethacin (below). FIG. 1E ACH dose response curve: Treatment with 5 μM COX-2 inhibitor (nimesulide), and 100 nM thromboxane receptor antagonist (SQ29548) enhanced endothelial dependent vasorelaxation in IL-10(tm/tm) aortas. FIG. 1F Endothelial counter vasoconstriction in IL-10(tm/tm) aortas at a dose of 1 μM is abolished on treatment with COX-2 and TxA2 antagonists.

FIGS. 2A-2E show noninvasive arterial stiffness and invasive carotid artery pressures measured in old IL-10(tm/tm) and WT mice. FIG. 2A The mean arterial pressure in old IL-10(tm/tm) mice is 89±18.6 mmHg as compared to age matched WT mice, 68±6.5 mmHg FIG. 2B Pulse wave velocity recorded at a heart rate of approximately 500 BPM is higher in old IL-10(tm/tm) as compared to the WT controls (3.72±0.12 m/s vs. 3.23±0.15 m/s). FIG. 2C COX2 mRNA measured via qPCR is higher in young IL-10(tm/tm) as compared to WT controls. FIG. 2D iNOS mRNA measured via qPCR is higher in young IL-10(tm/tm) as compared to WT controls. FIG. 2E Body mass (g) of young and old IL-10(tm/tm) and WT mice.

FIG. 3A Echocardiography images from old WT (left) and IL-10(tm/tm) (right) mice. Graphical representation of echocardiography and Doppler studies showing (FIG. 3B) Left ventricle end diastolic diameter (LVEDD), (FIG. 3C) Left ventricle end systolic diameter (LVESD), (FIG. 3D) Ejection fraction (EF), (FIG. 3E) Ratio of septal thickness (IVSD) and wall thickness (LVPWD) (FIG. 3F) Left ventricle mass (LV mass) and (FIG. 3G) Isovolumic relaxation time (IVRT), in young and old wild type and IL-10(tm/tm) mice.

FIG. 4A shows high resolution digitized images of H&E stained slides to determine myocyte cross-sectional diameter. FIG. 4B shows cardiac myocyte size measured in old WT and IL-10(tm/tm) groups.

### DETAILED DESCRIPTION OF THE INVENTION

The role of inflammatory pathway activation and elevation of serum inflammatory cytokines in age-related disease states, frailty, and functional decline is an active area of investigation. Chronic activation of NF-k6 induced inflammatory cascades, such as that induced via deletion of IL-10, influences the frailty phenotype and the associated vulnerability to multi-systemic decline in these mice, similar to that observed in frail older human adults. These conditions include hypertension, congestive heart failure, metabolic and endocrine abnormalities, among other conditions. Our efforts in the present invention were, in part, meant to determine whether the loss of IL-10 influences the cardiovascular pathophysiology observed in frailty and in aging, and help to determine if these changes may be a potential target for modifying age-related cardiovascular mortality and morbidity.

The studies of the present invention have established a relation between the loss of IL-10 and associated age related cardiovascular dysfunction. The inability of the aortas of old IL-10(tm/tm) mice to relax with muscarinic stimulation can be attributed to endothelial dysfunction. We also observed an increased blood pressure and vascular stiffness in old IL-10(tm/tm) as compared to age matched WT mice. Additionally, the hearts of the old IL-10(tm/tm) mice also undergo dynamic changes causing asymmetric hypertrophy, and both systolic and diastolic dysfunction.

The unchecked activation of endothelium causes activation of multiple signaling cascades. This especially includes the eicosanoids, the signaling molecules produced by the substrate arachidonic acid, specifically via prostaglandin H₂ (PGH₂) synthase (COX1/2 and peroxidase). These enzymes are committed to production of prostaglandins, prostacyclin and thromboxane. Different cell types convert PGH₂ to different end products, which may also depend on the cell stress and conditions. The peroxidase in PGH₂ synthase can produce peroxide, which oxidizes heme iron. The resulting heme is capable of accepting electron from tyrosine residue (385) and hence the resulting tyrosine residue is supposed to extract a hydrogen atom from arachidonic acid to produce reactive oxygen species. On the other hand, vascular endothelial cells express both isoforms of COX, COX-1 (constitutive) and COX-2 (inducible), which produce PGH₂, a substrate for both PGI₂ and TXA₂. While PGI₂ causes vasorelaxation, TXA₂ causes vasoconstriction. Indeed, the present invention shows the potential beneficial effect of COX inhibitors in endothelial protection as humans age.

In accordance with an embodiment, the present invention shows that IL-10 is more than just the cytokine synthesis inhibitory factor; it appears to contain and check the unregulated production of eicosanoids and their activation in response to local inflammatory processes such as in infection, systemic conditions like sepsis and chronic inflammatory processes such as aging. The frail and immune compromised phenotype of IL-10(tm/tm) mouse model reinforces the same. Unexpressed under most normal conditions and inducible under inflammatory stress, COX-2 is known to be nitrosylated and activated via iNOS, and IL-10 decreases TNF and iNOS production. Hence, in accordance with the present invention, IL-10 is thought to have the ability to suppress the activity of COX-2 by checking NOS activation. Indeed, the studies provided herein show that in youth the abundance of iNOS mRNA is 2 fold higher in the aortic tissue of IL-10 depleted mice as compared to WT controls. Similarly, this iNOS induction is able to drive the abundance of COX-2 mRNA, which is also significantly higher in young IL-10(tm/tm) mouse aorta as compared to WT counterparts.

In accordance with an embodiment, the present invention provides a method for screening biologically active agents which modulate IL-10 related effects on cardiovascular tissue comprising: a) providing test cardiovascular tissue from a IL-10(tm/tm) mouse and control cardiovascular tissue from a WT mouse; b) contacting the biologically active agent with both the test and control cardiovascular tissue for a sufficient period of time; c) measuring the effect of the biologically active agent on both the test and control cardiovascular tissue; wherein the effect being measured is selected from the group consisting of vasorelaxation, mean arterial blood pressure, pulse wave velocity, COX-2 mRNA expression, iNOS mRNA expression, left ventricular end-systolic diameter (LVESD) ejection fraction (EF), intraventricular septal thickness at end of diastole/left ventricular posterior wall thickness at end of diastole (IVSD/LVPWD) ratio, left ventricular (LV) mass, myocyte size and isovolumic relaxation time (IVRT); d) comparing the effect of the biologically active agent on both the test and control cardiovascular tissue, wherein when the effect of the biologically active agent on the test tissue is significantly different from the effect of the biologically active agent on the control tissue, identifying the biologically active agent as modulating IL-10 related effects on cardiovascular tissue.

In accordance with an embodiment, the inventive method for screening for biologically active agents measures vasorelaxation, and wherein when the vasorelaxation in the test tissue is equal to, or greater than the vasorelaxation in the control tissue, then the biologically active agent is identified as having a positive effect.

In another embodiment, the method for screening for biologically active agents measures mean arterial blood pressure, and wherein when the mean arterial blood pressure in the test tissue is equal to, or less than the mean arterial blood pressure in the control tissue, then the biologically active agent is identified as having a positive effect.

In a further embodiment, the method for screening for biologically active agents measures pulse wave velocity, and wherein when the pulse wave velocity in the test tissue is equal to, or less than the pulse wave velocity in the control tissue, then the biologically active agent is identified as having a positive effect.

In a still another embodiment, the method for screening for biologically active agents measures COX-2 mRNA expression, and the test tissue is young test tissue, wherein when the COX-2 mRNA expression in the test tissue is equal to, or less than the COX-2 mRNA expression in the control tissue, then the biologically active agent is identified as having a positive effect.

In an yet a further embodiment, the method for screening for biologically active agents measures iNOS mRNA expression, and the test tissue is young test tissue, wherein when the iNOS mRNA expression in the test tissue is equal to, or less than the iNOS mRNA expression in the control tissue, then the biologically active agent is identified as having a positive effect.

In an embodiment, the method for screening for biologically active agents measures LVESD and, wherein when the LVESD in the test tissue is equal to, or less than the LVESD in the control tissue, then the biologically active agent is identified as having a positive effect.

In another embodiment, the method for screening for biologically active agents measures EF and, wherein when the EF in the test tissue is equal to, or greater than the EF in the control tissue, then the biologically active agent is identified as having a positive effect.

In a still another embodiment, the method for screening for biologically active agents measures the IVSD/LVPWD ratio and, wherein when the IVSD/LVPWD ratio in the test tissue is equal to, or less than the IVSD/LVPWD ratio in the control tissue, then the biologically active agent is identified as having a positive effect.

In an yet a further embodiment, the method for screening for biologically active agents measures LV mass and, wherein when the LV mass in the test tissue is equal to, or less than the LV mass in the control tissue, then the biologically active agent is identified as having a positive effect.

In a still another embodiment, the method for screening for biologically active agents measures myocyte size and, wherein when the myocyte size in the test tissue is equal to, or less than the myocyte size in the control tissue, then the biologically active agent is identified as having a positive effect.

In yet another embodiment, the method for screening biologically active agents measures IVRT and, wherein when the IVRT in the test tissue is equal to, or less than the IVRT in the control tissue, then the biologically active agent is identified as having a positive effect.

As used herein, the term “positive effect” is intended to mean that the biologically active agents ameliorate, reduce or prevent the symptoms or physical or cellular effects present in aging and/or young IL-10(tm/tm) mice as compared to WT mice.

Here the present invention demonstrates a significantly greater increase in blood pressure and vascular stiffness in aging IL-10(tm/tm) mice as compared to WT mice. Thus, it is thought that the loss of compliance may not be a direct effect of IL-10 depletion but an effect of rise in blood pressure caused by endothelial dysfunction.

In accordance with an embodiment, the present invention provides a method to reduce, prevent, or delay age-related vascular stiffness in a subject comprising administrating to the subject a therapeutically effective amount of IL-10, or an IL-10 receptor agonist

Therefore, in accordance with another embodiment, the present invention provides a method to reduce, prevent, or delay age-related vascular stiffness in a subject comprising administrating to the subject a pharmaceutical composition comprising a therapeutically effective amount of IL-10, or an IL-10 receptor agonist and a pharmaceutically acceptable carrier.

It will be understood by those of ordinary skill in the art, that the pharmaceutical composition comprising a therapeutically effective amount of IL-10, or an IL-10 receptor agonist can also include one or more additional therapeutic agents and a pharmaceutically acceptable carrier.

An active agent, therapeutic agent, and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The term “ligand” refers to molecules, usually members of the family of cytokine-like peptides that bind to the receptor via the segments involved in peptide ligand binding. Also, a ligand is a molecule which serves either as a natural ligand to which the receptor, or an analog thereof, binds, or a molecule which is a functional analog of a natural ligand. The functional analog may be a ligand with structural modifications, or may be a wholly unrelated molecule which has a molecular shape which interacts with the appropriate ligand binding determinants. The ligands may serve as agonists or antagonists, see, e.g., Goodman, et al. (eds.) (1990) Goodman & Gilman's: The Pharmacological Bases of Therapeutics (8th ed.), Pergamon Press.

As used herein, the term “agonist” has its usual meaning and in general, means a biologically active agent which binds a receptor, for example, an IL-10 receptor, and activates the receptor resulting in a biological response.

“Treating” or “treatment” is an art-recognized term which includes curing as well as ameliorating at least one symptom of any condition or disease. Treating includes reducing the likelihood of a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing any level of regression of the disease; inhibiting the disease, disorder or condition, e.g., impeding its progress; and relieving the disease, disorder or condition, even if the underlying pathophysiology is not affected or other symptoms remain at the same level.

“Prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl)aminoethane; and the like, see, for example, J. Pharm. Sci., 66: 1-19 (1977).

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released the subject composition, for example, into adjacent tissues or fluids upon administration to a subject. In some embodiments, a biologically active agent may be used in cross-linked polymer matrix of this invention, to, for example, promote cartilage formation. In other embodiments, a biologically active agent may be used in cross-linked polymer matrix of this invention, to treat, ameliorate, inhibit, or prevent a disease or symptom, in conjunction with, for example, promoting cartilage formation.

Further examples of biologically active agents include, without limitation, enzymes, receptor antagonists or agonists, hormones, growth factors, autogenous bone marrow, antibiotics, antimicrobial agents, and antibodies.

Non-limiting examples of biologically active agents include following: adrenergic blocking agents, anabolic agents, androgenic steroids, anti-cholesterolemic and anti-lipid agents, anti-cholinergics and sympathomimetics, anti-coagulants, anti-hypertensive agents, anti-inflammatory agents such as steroids, non-steroidal anti-inflammatory agents, anti-pyretic and analgesic agents, anti-spasmodic agents, anti-thrombotic agents, anti-anginal agents, biologicals, cardioactive agents, coronary dilators, diuretics, diagnostic agents, erythropoietic agents, peripheral vasodilators, prostaglandins, stimulants, and prodrugs.

More specifically, non-limiting examples of useful biologically active agents include the following therapeutic categories: nonsteroidal anti-inflammatory drugs, salicylates; H₁-blockers and H₂-blockers; parasympathomimetics, cholinergic agonist parasympathomimetics, cholinesterase inhibitor parasympathomimetics, sympatholytics, a-blocker sympatholytics, sympatholytics, sympathomimetics, and adrenergic agonist sympathomimetics; cardiovascular agents, such as antianginals, antianginals, calcium-channel blocker antianginals, nitrate antianginals, antiarrhythmics, cardiac glycoside antiarrhythmics, class I antiarrhythmics, class antiarrhythmics, class antiarrhythmics, class IV antiarrhythmics, antihypertensive agents, a-blocker antihypertensives, angiotensin-converting enzyme inhibitor (ACE inhibitor) antihypertensives, 13-blocker antihypertensives, calcium-channel blocker antihypertensives, central-acting adrenergic antihypertensives, diuretic antihypertensive agents, peripheral vasodilator antihypertensives, antilipemics, bile acid sequestrant antilipemics, reductase inhibitor antilipemics, inotropes, cardiac glycoside inotropes, and thrombolytic agents; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; hematological agents, such as antianemia agents, hematopoietic antianemia agents, coagulation agents, anticoagulants, hemostatic coagulation agents, platelet inhibitor coagulation agents, thrombolytic enzyme coagulation agents, and plasma volume expanders; corticosteroid anti-inflammatory agents, gold compound anti-inflammatory agents, immunosuppressive anti-inflammatory agents, nonsteroidal anti-inflammatory drugs, salicylate anti-inflammatory agents, skeletal muscle relaxants, neuromuscular blocker skeletal muscle relaxants, and reverse neuromuscular blocker skeletal muscle relaxants.

Still further, the following listing of peptides, proteins, and other large molecules may also be used, such as interleukins 1 through 18, including mutants and analogues; interferons α, γ, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-a-y-globulin; superoxide dismutase (SOD); and complement factors, and biologically active analogs, fragments, and derivatives of such factors, for example, growth factors.

Members of the transforming growth factor (TGF) supergene family, which are multifunctional regulatory proteins, may be incorporated in a polymer matrix of the present invention. Members of the TGF supergene family include the beta transforming growth factors (for example, TGF-131, TGF-132, TGF-133); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)), (for example, inhibin A, inhibin B), growth differentiating factors (for example, GDF-1); and Activins (for example, Activin A, Activin B, Activin AB). Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

In certain embodiments, other materials may be incorporated into subject compositions in addition to one or more biologically active agents. For example, plasticizers and stabilizing agents known in the art may be incorporated in compositions of the present invention. In certain embodiments, additives such as plasticizers and stabilizing agents are selected for their biocompatibility or for the resulting physical properties of the reagents, the setting or gelling matrix or the set or gelled matrix.

Buffers, acids and bases may be incorporated in the compositions to adjust pH. Agents to increase the diffusion distance of agents released from the composition may also be included.

The charge, lipophilicity or hydrophilicity of a composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

The specific method used to formulate the novel formulations described herein is not critical to the present invention and can be selected from a physiological buffer (Feigner et al., U.S. Pat. No. 5,589,466 (1996)).

Therapeutic formulations of the product may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

The compositions can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

Isotonicifiers are present to ensure physiological isotonicity of liquid compositions of the instant invention and include polhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on.

Additional miscellaneous excipients include bulking agents, (e.g., starch), chelating agents (e.g., EDTA), antioxidants (e.g., ascorbic acid, methionine or vitamin E) and cosolvents.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml.

The present invention provides liquid formulations of a biopolymer having a pH ranging from about 5.0 to about 7.0, or about 5.5 to about 6.5, or about 5.8 to about 6.2, or about 6.0, or about 6.0 to about 7.5, or about 6.5 to about 7.0.

The incubation of the amine-reacting proteoglycan with blood or tissue product can be carried out a specific pH in order to achieve desired properties. E.g., the incubation can be carried out at between a pH of 7.0 and 10.0 (e.g., 7.5, 8.0, 8.5, 9.0, and 9.5). Furthermore, the incubation can be carried out for varying lengths of time in order to achieve the desired properties.

The instant invention encompasses formulations, such as, liquid formulations having stability at temperatures found in a commercial refrigerator and freezer found in the office of a physician or laboratory, such as from about 20° C. to about 5° C., said stability assessed, for example, by microscopic analysis, for storage purposes, such as for about 60 days, for about 120 days, for about 180 days, for about a year, for about 2 years or more. The liquid formulations of the present invention also exhibit stability, as assessed, for example, by particle analysis, at room temperatures, for at least a few hours, such as one hour, two hours or about three hours prior to use.

Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10%

The formulations to be used for in vivo administration must be sterile. That can be accomplished, for example, by filtration through sterile filtration membranes. For example, the formulations of the present invention may be sterilized by filtration.

### EXAMPLES

Animals: Age-matched IL-10 deficient (IL-10(tm/tm)); B6.129P2IL10tm1Cgn/J and control mice (C576L6; WT) were obtained from Jackson Laboratories (Bar Harbor, Me., USA). IL-10(tm/tm) mice used are homozygous for the IL10tm1Cgn targeted mutation. These mice were housed in Association for Assessment and Accreditation of Laboratory Animal Care International accredited facilities and pathogen contact prevention (prophylaxis from infections, inflammatory bowel disease and early mortality) was achieved under specific pathogen-free (SPF) barrier conditions until terminal experiments were carried out. It is known that the pro-inflammatory potential achieved by the lack of IL-10 in this mouse model can be attributed to activation of TNF-α and IL-1β synthesis via IFN-γ, which is produced in massive amounts and also is important inantigen presentation and pathogen death via activation of macrophages. Animals with any signs of inflammatory/infectious disease were ruled out of the study. The study was performed at approximately 3-4 months (young) and 9 months of age or greater (old).

Vascular endothelial function was assessed using force-tension myography. Mouse aortas were isolated and cleaned in ice-cold Krebs-Ringer-bicarbonate solution containing the following (in mM): 118.3 NaCI, 4.7 KCI, 1.6 CaCl2, 1.2 KH2 PO4, 25 NaHCO3, 1.2 MgSO₄, and 11.1 dextrose. Vascular tension changes were determined as previously described (J Appl Physiol, 2000. 89(6): p. 2382-90). Briefly, one end of the aortic rings was connected to a transducer, and the other to a micromanipulator. The aorta was immersed in a bath filled with constantly oxygenated Krebs buffer at 37° C. Equal size thoracic aortic rings (2 mm) were mounted using a microscope, ensuring no damage to the smooth muscle or endothelium. The aortas were passively stretched to an optimal resting tension using the micromanipulator, after which a dose of 60 mM KCI was administered, and repeated after a wash with Krebs buffer. After these washes, the vessels were allowed to equilibrate for 20-30 min. Phenylephrine (1 μM) was administered to induce vasoconstriction. A dose-dependent response (1 nM to 10 μM), with the muscarinic agonist, ACH, was then performed. The responses were repeated in the presence of inhibitors. Relaxation responses were calculated as a percentage of tension following pre-constriction. Sigmoidal dose-response curves were fitted to data with the minimum constrained to 0.

Pulse Wave Velocity (PWV) was measured non-invasively using a high-frequency, high-resolution Doppler spectrum analyzer (DSPW). Mice were anesthetized with 1.5% Isoflurane, placed supine on the heated (37° C.) plate. The animals were maintained at a physiologic heart rate of approximately 500. 10 MHz probe was used to record the aortic pulse waves at thorax and abdomen separately at a distance of 4 cm. EKG was recorded simultaneously and the time taken by the wave to reach from thoracic aorta to abdominal aorta was measured using R wave of the EKG as a fixed point. Subsequently, the velocity was calculated.

Blood Pressures were measured invasively through high fidelity solid-state transducer. The animals were anesthetized using 1.5-2% isoflurane for induction of anesthesia and then maintained at 1%. A midline neck skin incision was made and blunt dissection was carried out to access, clean and catheterize jugular vein for the purpose of saline infusion. Similarly carotid artery was catheterized with 1.2 F Scisence Pressure CatheterTTM. Data was recorded and analyzed using ADlnstrument Labchart version 7.

Real Time Quantitative Polymerase Chain Reaction from isolated mice aortas using Trizol and RNeasy previously (Am J Physiol Heart Circ Physiol, 2012. 302(10): p. H1919-28). RNA was then reverse transcribed cDNA using Superscript First Strand kit (Invitrogen (Applied Biosystems) was performed using SYBR Biosystems) and the following primer sets: COX2: forward 5′ ACACACTCTATCACTGGCACC (SEQ ID NO: 1); COX2: reverse 5′ CAAACTGAGTGAGTCCATGTT (SEQ ID NO: 2); iNOS: forward 5′ GGCTTGCCCCTGGAAGTTTCTCTTCAA AGT C (SEQ ID NO: 3); iNOS: reverse 5′ AAGGAGCCATAATACTGGTTGATG (SEQ ID NO: 4); 18s: forward 5′ AGAAACGGCTACCACATCCAA (SEQ ID NO: 5); 18s: reverse 5′ GGGTCGGGAGTGGGTAATTT (SEQ ID NO: 6).

Transthoracic Echocardiography in conscious mice was performed using Sequoia Acuson C256 (Malvern, Pa.) ultrasound machine, equipped with a frequency bandwidth of 15 MHz (Am J Physiol, 1999. 277(5 Pt 2): p. H1967-74; Cancer Res, 2003. 63(20): p. 6602-6). The two-dimensional (2-D) and M-mode echocardiogram were obtained in the parasternal short and long axis view of the left ventricle (LV) at the level of the papillary muscles and sweep speed of 200 mm/sec. Using the M-mode echocardiogram image, four parameters were measured: (i) left ventricular posterior wall thickness at end of diastole (LVPWD), (ii) interventricular septa) thickness at end of diastole (IVSD), (iii) left ventricle (LV) chamber diameter at end of diastole (LVEDD), and (iv) left ventricle chamber diameter at end of systole (LVESD). All measurements were performed according to the guidelines set by the American Echocardiography Society. For each mouse, three to five values for each measurement were obtained and averaged for evaluation. Using the LVEDD and LVESD, we derived the fractional shortening (FS) which represented the percent change in left ventricular (LV) chamber dimension with systolic contraction. We used the FS in the estimation of the LV wall contractility or the systolic function based on the following equation: FS (%)=[(LVEDD−LVESD)/LVEDD]×100 The left ventricular mass (LVmass) was derived and used in the assessment of left ventricular hypertrophy and enlargement, using the following equation: LV mass (mg): 1.055 [(IVSD+LVEDD+pWTED)³−(LVEDD)³] where 1.055 is the specific gravity of the myocardium (J. Am. Soc. Echocardiogr., 1995 8(5 Pt 1): p. 602-10).

Doppler Imaging: Doppler imaging was used for evaluation of regional wall motion. Myocardial relaxation (diastolic) and contraction (systolic) velocities of the left ventricle were measured using the four-chamber view. The sample volume was positioned at the basal level of the inter-ventricular septum. The isovolumetric relaxation time (IVRT) was measured as an index of diastolic function. All measurements were performed according to the guidelines set by the American Society of Echocardiography. For each mouse, three to five values for each measurement were obtained and averaged for evaluation.

Histological evaluation and cellular morphometry: Myocardium was fixed in 10% formalin, processed by standard paraffin embedding and serially sectioned in 5-8 μm thicknesses. Myocyte cross-sectional diameter was determined from digitized images of hematoxylin and eosin (H&E) stained slides and analyzed using Image 1 program (NIH, Bethesda, Md.).

Statistical analysis. The results were expressed as mean and standard error (mean±SEM). One-way analysis of ANOVA and the Bonferroni post hoc test for multiple-comparison were used for comparing all groups and pairs of groups respectively. A P<0.05 was considered significantly different. All analyses were carried out using Graph Pad version 5 and Microsoft Excel version 14.1.3 statistical analysis software.

### Example 1

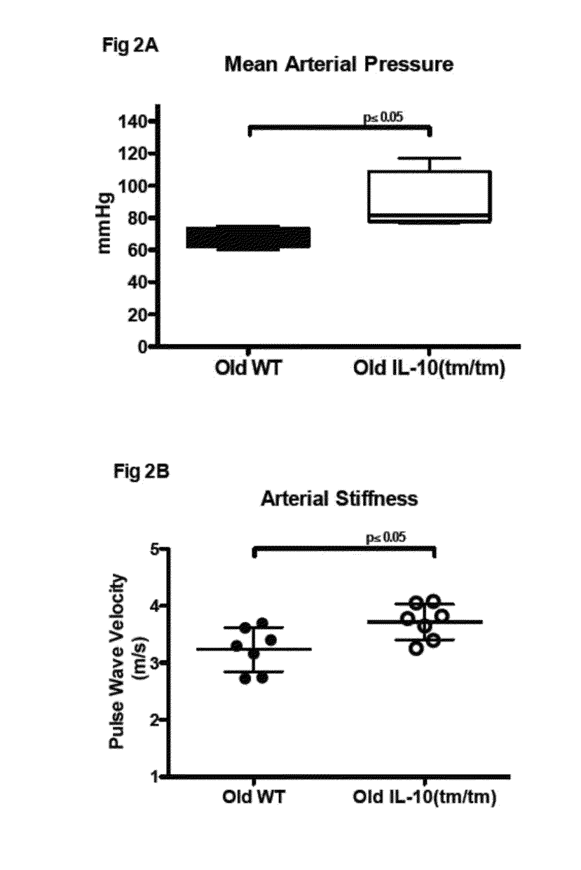

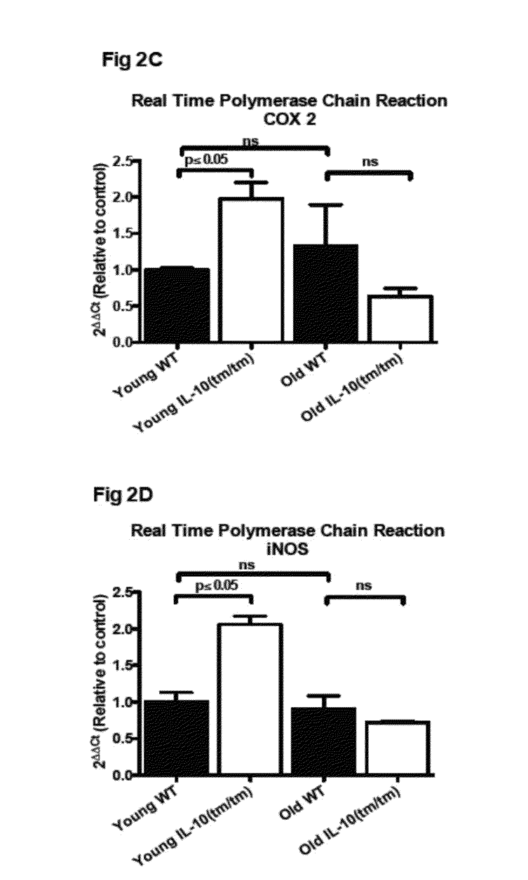

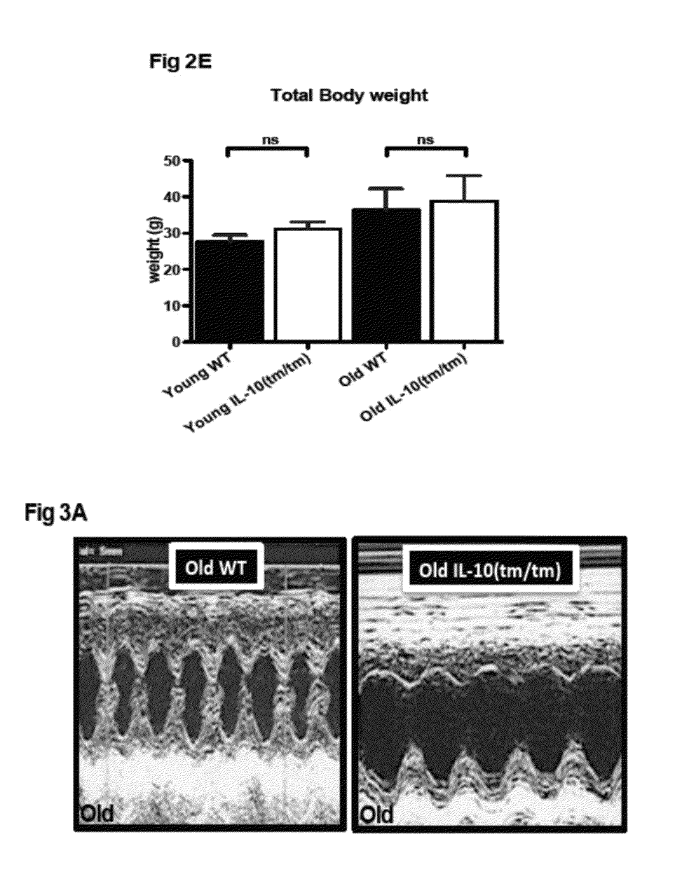

Body Mass. There was no significant difference in the body mass in aye matched IL-10(tm/tm and WT mice. Young IL-10(tm/tm) vs. WT mice average weight was measured to be 27 g vs. 31 g and in old IL-10(tm/tm) vs. WT mice group the average weights were 38 g vs. 36 g (FIG. 2E).

### Example 2

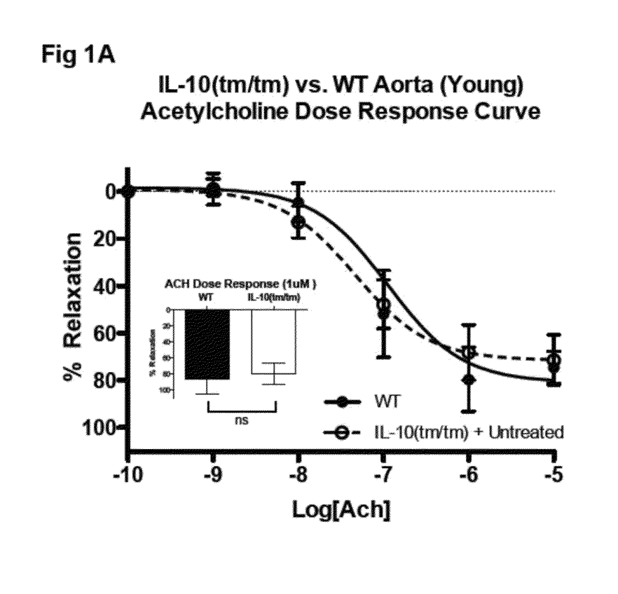

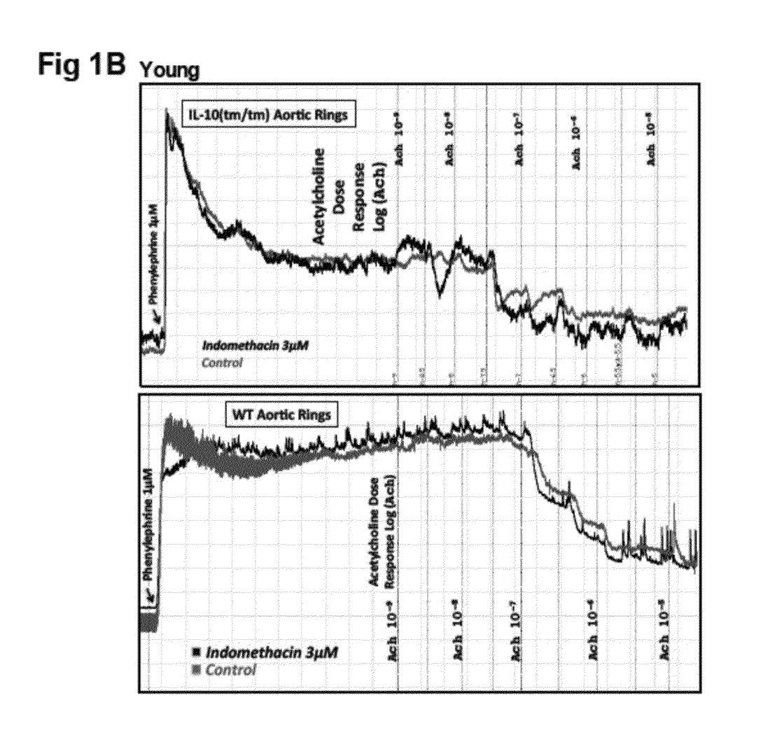

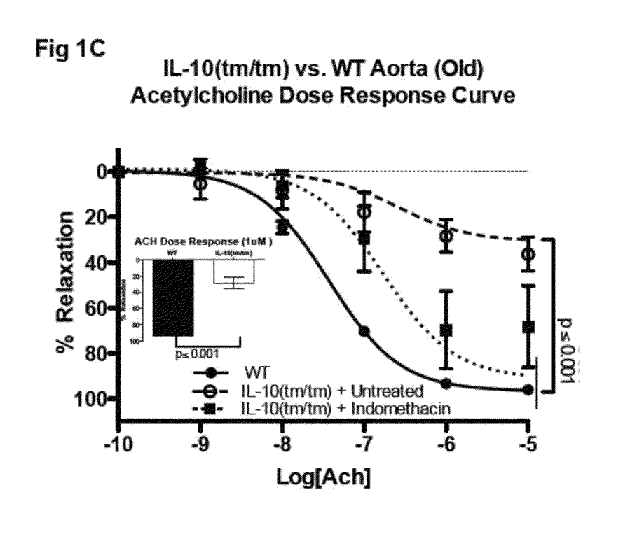

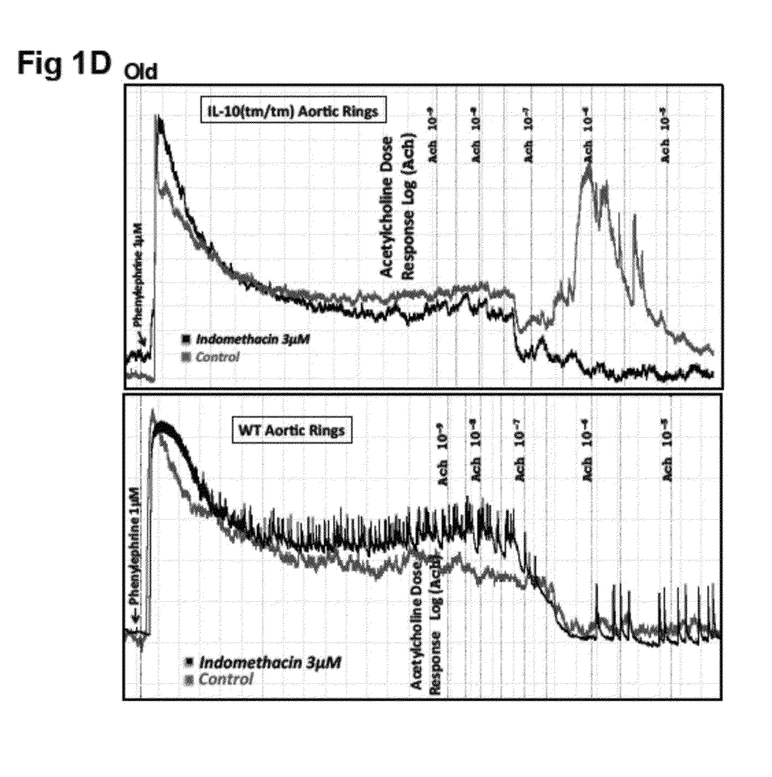

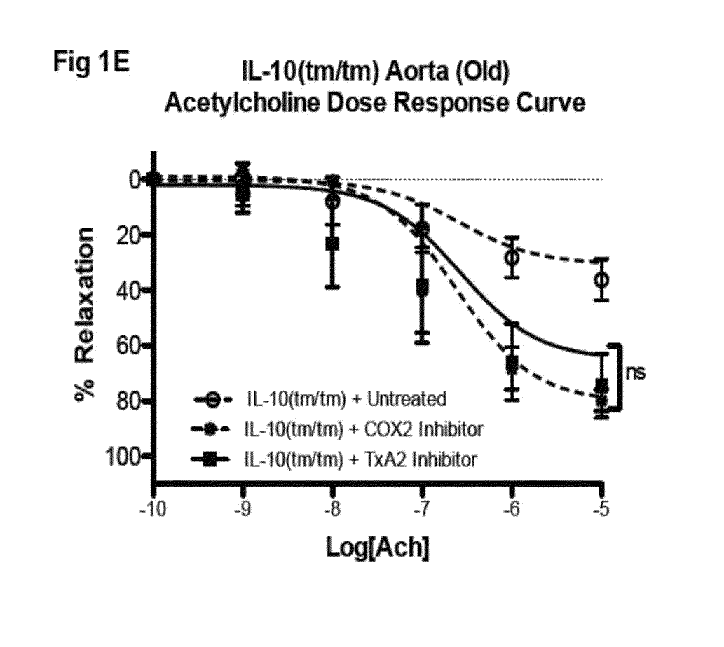

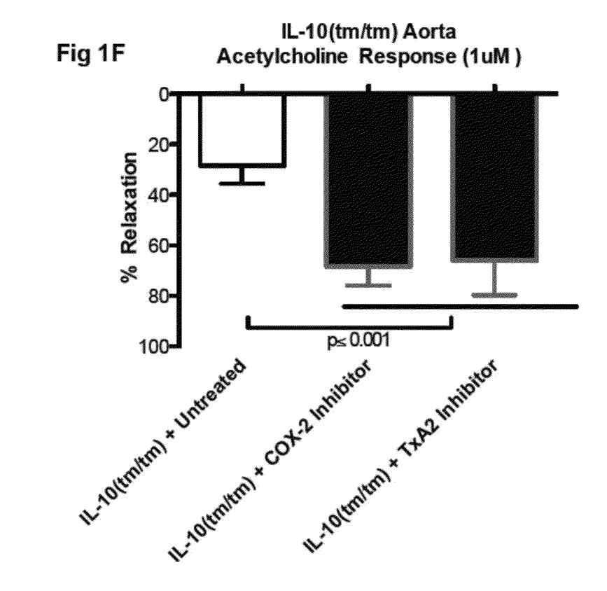

Vascular Studies. In ex vivo myograph experiments, measured tension represents a balance between vasorelaxant and vasoconstrictor dependent function and mediators. In phenylephrine pre-constricted isolated mouse aorta, ACH stimulates the release of endothelial factors, which mediate vasorelaxation as a result of greater relaxation than constriction. In young animals the ACH dose response curves were no different in aortas from WT as compared to IL-10(tm/tm) (Eₘₐₓ, 80.9±4.6 vs. 71.9±5.7%; EC₅₀ 125.9 nM vs 50.1 nM) in IL-10(tm/tm) mice aortas (FIG. 1A). By contrast, in old mice ACH mediated vasorelaxation was markedly impaired in IL-10 as compared to WT age matched controls (Eₘₐₓ 30.7±9.3 vs. 98.5±14.1%; EC₅₀ 39.4 nM vs. 251 nM; p<0.001, n=6) (FIG. 1C). Furthermore vasoconstriction was observed at higher doses (>1 μM) of ACH in old IL-10 aortas (FIGS. 1C, 1D).

Pre-incubation of aortic rings with 3 μM indomethacin (COX1/2 inhibitor), 5 μM COX-2 inhibitor (nimesulide), or 100 nM thromboxane receptor antagonist (SQ29548) abolished the vasoconstrictive responses and significantly improved endothelial dependent vasorelaxation in old IL-10(tm/tm) aortas (Emax 80.3±2.6%, 82.9±2.0%, 65.1±3.2%; EC₅₀ 171 nM, 240 nM, 265 nM respectively) (FIGS. 1E, 1F).

### Example 3

Mean arterial blood pressure (MAP) was significantly increased in old IL-10(tm/tm) mice as compared to WT age matched controls (89±18.6 mmHg vs. 68±6.5 mmHg, p<0.05, n=4; FIG. 2A). Furthermore PWV a measure of vascular stiffness was also significantly increased in old IL-10(tm/tm) mice as compared to WT mice (3.72±0.12m/s vs. 3.23±0.15m/s, p<0.05, n=7) (FIG. 2B). There was no significant difference observed in the PWVs of young WT and IL-10(tm/tm) mice.

### Example 4

The abundance of COX2 mRNA was significantly increased in aortas of young IL-10(tm/tm) mice as compared to WT age matched controls (1.97±0.13 2^{ΔΔct} vs.0.99±0.02 2^{ΔΔc}. p<0.05, N=6). There was no statistical difference in abundance of COX2 mRNA in old age matched IL-10(tm/tm) mice as compared to WT aortas (0.63±0.06 2^{ΔΔct} vs. 1.33±0.32 2^{ΔΔct} ns, N=6) (FIG. 2C).

### Example 5

The abundance of iNOS mRNA was significantly increased in aortas of young IL-10(tm/tm) mice as compared to WT age matched controls (2.06±0.06 2^{ΔΔct} vs. 1.00±0.07 2^{ΔΔc}. p<0.05, N=6). There was no statistical difference in abundance of iNOS mRNA in old age matched IL-10(tm/tm) mice as compared to WT aortas (0.72±0.01 2^{ΔΔct} vs. 0.90±0.10 2^{ΔΔct} ns, N=61 (FIG. 2D).

### Example 6

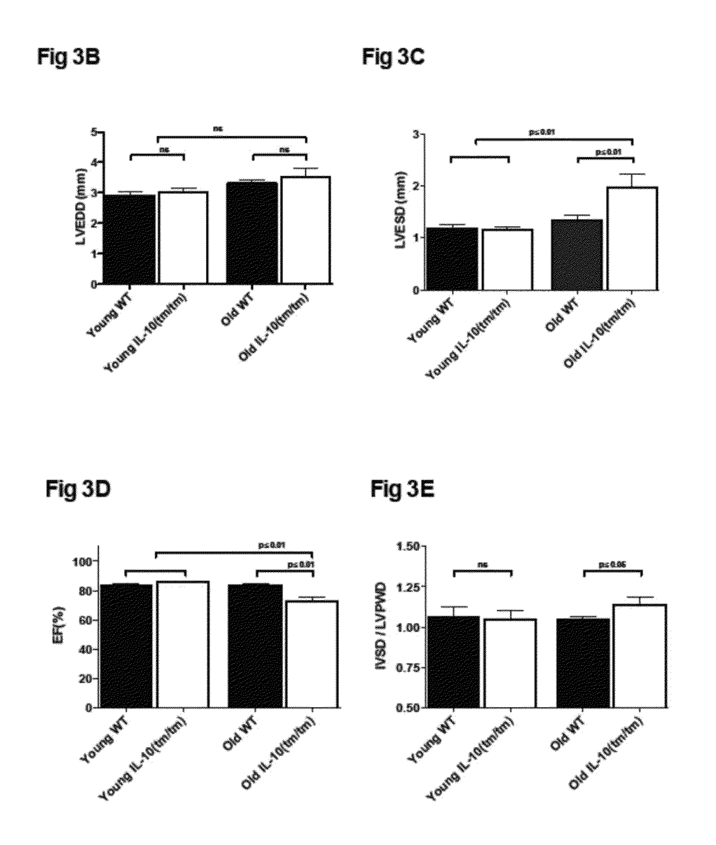

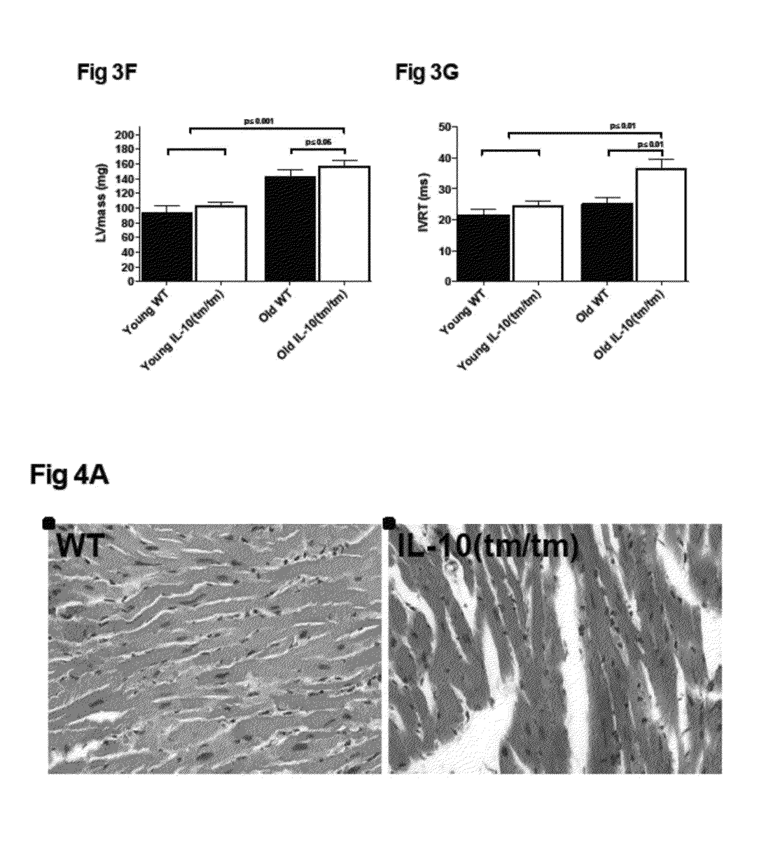

Cardiac echocardiography (FIGS. 3A-3F) demonstrated no difference in LVEDD between the old IL-10(tm/tm) (3.5±0 2 mm) as compared to old WT and young WT and IL-10(tm/tm) mice groups (3.3±0.1mm, 2.9±0.1mm, 3.0±0.1mm respectively). In contrast, left ventricular end-systolic diameter (LVESD) was significantly greater in old IL-10(tm/tm) mice (2.0±0.2 mm) as compared to age matched WT (1.5±0.1 mm), and young WT (1.2±0.09 mm) and IL-10(tm/tm) (1.2±0.06 mm) mice (p<0.01, n=7) (FIG. 3C).

A significant reduction in ejection fraction (EF) was also observed in old IL-10(tm/tm) mice (73±3%) as compared to old WT (84±1%; p<0.01) mice, and young WT (84±1%; p<0.01) and IL-10(tm/tm) (86±1%; p<0.001) mice (n=7) (FIG. 3D).

WT hearts undergo symmetric changes with no difference in IVSD/LVPWD ratio between young and old WT mice (IVSD/LVPWD=1.06 vs. 1.04). Aging of IL-10(tm/tm) mice results in asymmetric cardiac hypertrophy; IVSD/LVPWD in old IL-10(tm/tm) mice is significantly higher than young IL-10(tm/tm) mice (IVSD/LVPWD=1.14 vs. 1.05; p<0.05, n=7) (FIG. 3E).

LV mass was significantly increased in the old IL-10(tm/tm) (156.3±9.0 mg) as compared old WT (142.2±10.1 gm; p<0.05) and young WT (92.9±10.5 gm; p<0.001) and IL-10(tm/tm) (102.3±5.1; p<0.001) mice, suggesting LVESD dilatation and heart enlargement (n=7); (FIG. 3F).

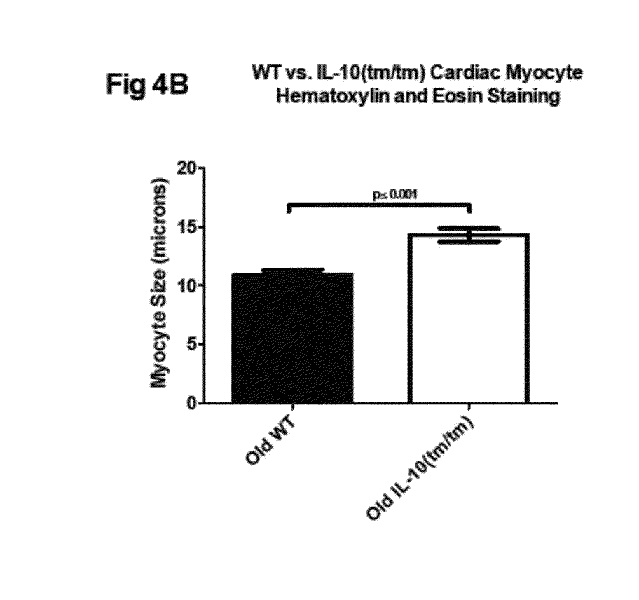

Also, H&E staining in old mice demonstrated an increase in myocyte size in IL-10(tm/tm) group as compared to age matched WT controls (14.3±3.7 μm vs. 10.9±2.8 μm; p<0.001, n=45) (FIGS. 4A-4B).

Isovolumic relaxation time (IVRT), an index of diastolic function, was significantly increased in old IL-10(tm/tm) mice (36.3±3.4 ms) as compared to age matched WT controls (25.0±2.0 ms) and young WT (21.50±1.89 ms) and IL-10(tm/tm) mice (24.50±1.71 ms) (p<0.01, n=7) (FIG. 3G).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for screening biologically active agents which modulate IL-10 related effects on cardiovascular tissue comprising:
a) providing test cardiovascular tissue from a IL-10(tm/tm) mouse and control cardiovascular tissue from a WT mouse;
b) contacting the biologically active agent with both the test and control cardiovascular tissue for a sufficient period of time;
c) measuring the effect of the biologically active agent on both the test and control cardiovascular tissue; wherein the effect being measured is selected from the group consisting of vasorelaxation, mean arterial blood pressure, pulse wave velocity, COX-2 mRNA expression, iNOS mRNA expression, left ventricular end-systolic diameter (LVESD) ejection fraction (EF), intraventricular septal thickness at end of diastole/left ventricular posterior wall thickness at end of diastole (IVSD/LVPWD) ratio, left ventricular (LV) mass, myocyte size and isovolumic relaxation time (IVRT);
d) comparing the effect of the biologically active agent on both the test and control cardiovascular tissue, wherein when the effect of the biologically active agent on the test tissue is significantly different from the effect of the biologically active agent on the control tissue, identifying the biologically active agent as modulating IL-10 related effects on cardiovascular tissue.

2. The method of claim 1, wherein the effect is vasorelaxation and wherein when the vasorelaxation in the test tissue is equal to, or greater than the vasorelaxation in the control tissue, then the biologically active agent is identified as having a positive effect.

3. The method of claim 1, wherein the effect is mean arterial blood pressure and wherein when the mean arterial blood pressure in the test tissue is equal to, or less than the mean arterial blood pressure in the control tissue, then the biologically active agent is identified as having a positive effect.

4. The method of claim 1, wherein the effect is pulse wave velocity and wherein when the pulse wave velocity in the test tissue is equal to, or less than the pulse wave velocity in the control tissue, then the biologically active agent is identified as having a positive effect.

5. The method of claim 1, wherein the effect is COX-2 mRNA expression and the test tissue is young test tissue, wherein when the COX-2 mRNA expression in the test tissue is equal to, or less than the COX-2 mRNA expression in the control tissue, then the biologically active agent is identified as having a positive effect.

6. The method of claim 1, wherein the effect is iNOS mRNA expression and the test tissue is young test tissue, wherein when the iNOS mRNA expression in the test tissue is equal to, or less than the iNOS mRNA expression in the control tissue, then the biologically active agent is identified as having a positive effect.

7. The method of claim 1, wherein the effect is LVESD and, wherein when the LVESD in the test tissue is equal to, or less than the LVESD in the control tissue, then the biologically active agent is identified as having a positive effect.

8. The method of claim 1, wherein the effect is EF and, wherein when the EF in the test tissue is equal to, or greater than the EF in the control tissue, then the biologically active agent is identified as having a positive effect.

9. The method of claim 1, wherein the effect is IVSD/LVPWD and, wherein when the IVSD/LVPWD in the test tissue is equal to, or less than the IVSD/LVPWD in the control tissue, then the biologically active agent is identified as having a positive effect.

10. The method of claim 1, wherein the effect is LV mass and, wherein when the LV mass in the test tissue is equal to, or less than the LV mass in the control tissue, then the biologically active agent is identified as having a positive effect.

11. The method of claim 1, wherein the effect is myocyte size and, wherein when the myocyte size in the test tissue is equal to, or less than the myocyte size in the control tissue, then the biologically active agent is identified as having a positive effect.

12. The method of claim 1, wherein the effect is IVRT and, wherein when the IVRT in the test tissue is equal to, or less than the IVRT in the control tissue, then the biologically active agent is identified as having a positive effect.

13. A method to reduce, prevent, or delay age-related vascular stiffness in a subject comprising administrating to the subject a pharmaceutical composition comprising a therapeutically effective amount of IL-10, or an IL-10 receptor agonist.

14. The method of claim 13, wherein the pharmaceutical composition comprises at least one additional therapeutic agent.


SEQUENCE LISTING (6 sequences)

SEQ ID NO: 1 - DNA, 21 bp, Artificial Sequence
  synthetic primer
acacactctatcactggcacc

SEQ ID NO: 2 - DNA, 21 bp, artificial sequence
  synthetic primer
caaactgagtgagtccatgtt

SEQ ID NO: 3 - DNA, 31 bp, Artificial Sequence
  synthetic primer
ggcttgcccctggaagtttctcttcaaagtc

SEQ ID NO: 4 - DNA, 24 bp, Artificial Sequence
  synthetic primer
aaggagccataatactggttgatg

SEQ ID NO: 5 - DNA, 21 bp, Artificial Sequence
  synthetic primer
agaaacggctaccacatccaa

SEQ ID NO: 6 - DNA, 20 bp, Artificial Sequence
  synthetic primer
gggtcgggagtgggtaattt